(19) United States
(12) Patent Application Publication
(10) Pub. No.: US 2024/0390539 A1
(43) Pub. Date: Nov. 28, 2024

(54) DISINFECTION DEVICE FOR PLUMBING FIXTURES

(71) Applicant: Kohler Co., Kohler, WI (US)

(72) Inventor: Clayton Garrels, Sheboygan, WI (US)

(21) Appl. No.: 18/666,343

(22) Filed: May 16, 2024

Related U.S. Application Data
(60) Provisional application No. 63/469,272, filed on May 26, 2023.

(51) Int. Cl. A61L 2/18

(52) U.S. Cl. CPC A61L 2/183; A61L 2202/121; A61L 2202/14; A61L 2202/15

(57) ABSTRACT

A water disinfection apparatus includes an air pump, a chamber including a rod, a turbine connected to a water supply and configured to translate energy from the water supply to rotate the air pump and the rod, an ozone supply downstream of the air pump and connected to the chamber, and a water path from the turbine to the chamber.

### CROSS-REFERENCE TO RELATED APPLICATION

This application claims the benefit of and priority to U.S. Provisional Patent Application No. 63/469,272, filed May 26, 2023, which is incorporated herein by reference.

### FIELD

The present application relates generally to the disinfection of appliances including plumbing fixtures using treated water.

### BACKGROUND

Ozone, or trioxide or O₃, is an inorganic molecule and reactive gas. It may be pale blue in color and present a distinctive odor. It is an allotrope of oxygen and less stable than oxygen. Ozone may be formed naturally in the atmosphere by reaction with ultraviolet light from the sun and electrical discharges in the atmosphere.

Ozonated water is a powerful disinfectant that can be generated on-site. On-site generation has advantages. One advantage is that there are no consumable chemicals required. One problem is that ozone has a slow and limited solubility in water. As such, ozone gas likely escapes from the water surface and into the environment.

### BRIEF DESCRIPTION OF THE DRAWINGS

Exemplary embodiments are described herein with reference to the following drawings, according to an exemplary embodiment.

FIG. 1 illustrates an example disinfection system.

FIG. 2 illustrates an example series of reactions for the disinfection system of FIG. 1.

FIG. 3 illustrates an example disinfection system.

FIG. 4 illustrates an example second embodiment for the disinfection system.

FIG. 5 illustrates an example third embodiment for the disinfection system.

FIG. 6 illustrates an example fourth embodiment for the disinfection system.

FIG. 7 illustrates an example disinfection device including a catalyst chamber.

FIG. 8 illustrates a cross section of the device of FIG. 7.

FIG. 9 illustrates a cutaway view of the device of FIG. 7.

FIG. 10 illustrates another example disinfection device.

FIG. 11 illustrates a cutaway view of the device of FIG. 10.

FIG. 12 illustrates an example turbine drive shaft.

FIGS. 13A-D illustrate example application of the water disinfection system.

FIG. 14 illustrates a toilet including the water disinfection system.

FIG. 15 illustrates an in wall tank including the water disinfection system.

FIG. 16 illustrates a shower including the water disinfection system.

FIG. 17 illustrates a sink including the water disinfection system.

FIG. 18 illustrates an example controller for any of the water disinfection systems.

FIG. 19 illustrates an example flow chart for the controller of FIG. 18.

### DETAILED DESCRIPTION

Ozone and water are provided to a microbubble generator. Microbubbles are suspended in water. The microbubbles may be defined according to a size range. One example size for microbubbles is a spherical diameter of 50 microns or smaller. An energy device provides energy to aid in the collapse of the microbubbles. The collapse of the microbubbles may break down the ozone (O₃) into hydroxyls (OH—). The hydroxyls may have a short half-life and be highly reactive. The collapse of the microbubbles may additionally or alternatively include cavitation. A catalyst may also be applied to the water, after or substantially during the time that the microbubbles are collapsing. The catalyst may include an ultraviolet light and/or a catalyst coating. One or more additional reactions may take place in response to the catalyst. One key objective is to coordinate the time the microbubble collapses with the passage of water near the location of the catalyst.

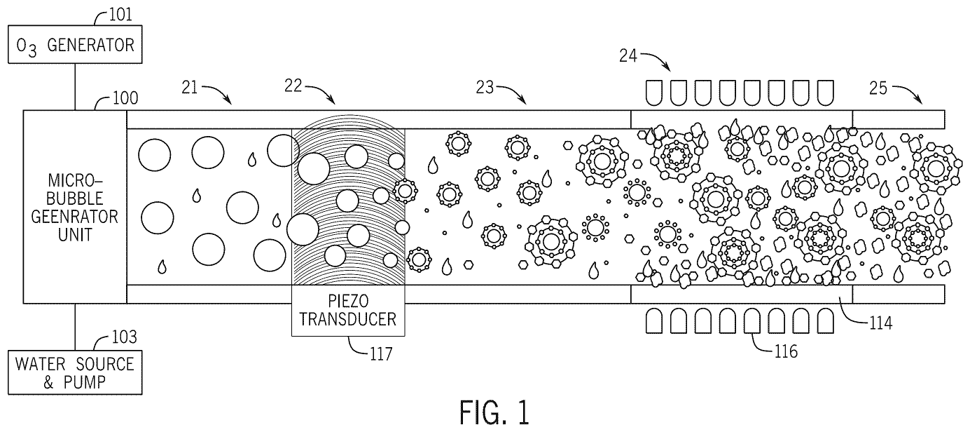

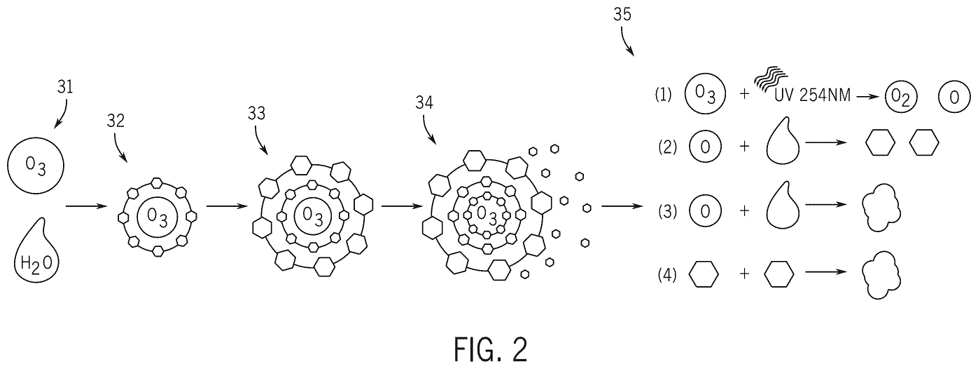

FIG. 1 illustrates an example disinfection system as a single path or pipe. FIG. 2 illustrates an example series of reactions for the disinfection system of FIG. 1. The pipe may be in-line, for example installed in a pipe of a plumbing system in a home, building or other facility. The disinfection system may be integrated with or coupled to any water consuming appliances such as toilets, bathtubs, bidets, drinking fountains, hose bibs, sinks, showers, faucets, shower heads, urinals, or other devices.

A microbubble generator unit 11 receives at least ozone from an ozone generator 101 and water from a water source 103. Ozone may be formed by the ozone generator 101 using a variety of techniques, including corona discharge, ultraviolet light, cold plasma, and other techniques, as described herein. The water source 103 may include a connection to a water source (line supply) of the building or facility that provides a flow of water under line pressure. The ozone generator 101 may include one or more heat sinks mounted on the outside of an ozone chamber where the ozone is generated and expels heat.

In a first region 21 the microbubbles of ozone are released from the microbubble generator (e.g., ozone generator 101). The microbubbles of ozone are suspended in water, as shown at 31 in FIG. 2. The microbubbles may be defined by a predetermined size such 50 microns or smaller.

In a second region 22, a transducer 117 applies energy to the water including the microbubbles. The transducer may be a piezoelectric transducer. The transducer may emit ultrasound waves. One example frequency of the ultrasound is 45 kilohertz (kHz). The energy (ultrasound) may initiate the microbubbles to collapse or implode in the pipe. While collapsing, the ozone O₃ is broken down creating hydroxyls OH— at the boundary of the microbubble, as shown as a first layer at 32 in FIG. 2 and a second layer 33 in FIG. 22.

In a third region 23, the microbubbles may ultimately completely or substantially completely collapse and disappear over a predetermined time, as shown at 34 in FIG. 2. The hydroxyl radicals are very reactive and may have a half live of several nanoseconds (e.g., 10 nanoseconds). Many hydroxyls are created from the microbubbles' collapse so that hydroxyls are present over a span of time (e.g., several seconds). Some relatively small amount of residual ozone O₃ may remain (i.e., not all microbubbles completely collapse).

In a fourth region 24, a catalyst 114 activated by a light 116 creates multiple reactions. The catalyst 1144 may include titanium oxide (TiO₂). The light 116 may emit ultraviolet light having a predetermined wavelength. For example, the light source 151 may be a UV-C lamp that produces light with a 254 nanometer wavelength. The UV-C lamp may produce light in an ozone destruction range such as 240-315 nanometer wavelength. As described herein, other wavelengths of ultraviolet light (e.g., 185 nanometer wavelength) may be harnessed to produce ozone. The catalyst 114 and/or light 116 causes multiple reaction at the collapsed bubbles as shown at 35 in FIG. 2.

In reaction (1), the ozone is converted to free oxygen gas and oxygen radicals.

In reaction (2) the oxygen radicals and water are converted to hydroxyls (OH—)

In reaction (3) the oxygen radicals and water are converted to peroxide (H₂O₂).

In reaction (4) the hydroxyls (OH—) are converted to peroxide (H₂O₂).

In a fifth region 25, the disinfectant water is output from the disinfection system. The key is to coordinate the microbubble collapse in time with the catalyst 114 and light 116 so the disinfectant characteristic is maximized at the output of the disinfectant water. The hydroxyls are able to break apart many different types of chemical compounds and may substantially eliminate bacteria and viruses.

One or more of the processes or reactions described with respect to FIGS. 1 and 2 are implemented by the following embodiments. The microbubble generator unit 11 may represent any of the devices described in the following embodiments.

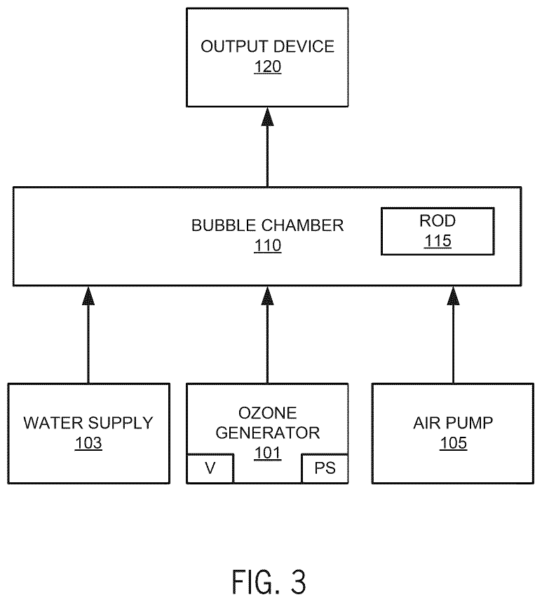

FIG. 3 illustrates an example disinfection system including a bubble chamber 110 including a rod 115 is connected to an ozone generator 101, a water supply 103, and an air pump 105. Additional, different, or fewer components may be included.

The rod 115 may be formed of graphite. The rod 115 may include one or more layers of carbon. The rod 115 may be at least partially hollow such that an input to the rod 115 is connected to a passage inside of the rod 115. The air pump 105 provides air and the ozone generator 101 provides ozone, which are combined at the input to the rod 115 and provided to the interior passage of the rod 115.

The water supply 103 may be a line supply connected to the plumbing system of the building. The water supply 103 alternatively may include a water tank. The water tank may be fed to the water supply 103 using gravity. The water tank may include a water tube for providing the water via the water supply 103. The water tank may include a pump for pumping the water to the water supply 103. The pump may be manual or electrically powered.

The air pump 105 provides a flow of air under pressure. The pressure may be greater than the atmospheric pressure in the vicinity of the disinfection system. Some example pumps include impellors. Others may include submersible pumps, displacement pumps, diaphragm pumps, or piston pumps.

The ozone generator 101 is configured to generate ozone using at least one of a variety of techniques, including corona discharge, ultraviolet light, cold plasma, and other techniques. For example, a corona charger (e.g., corona wire) may be configured to accumulate electric charge from a power source PS and apply the electric charge to air supplied through a vent V. The vent V may be open to the atmosphere surrounding the disinfection system. The vent V may be connected via a tube to a predetermined location.

In corona discharge, a corona wire, a corona discharge tube, or an ozone plate is used in the ozone generator 101. For example, a high voltage may be applied to an electrode in the corona wire, the discharge tube or on the ozone plate. A corona discharge is an electrical discharge caused by the ionization of air surrounding the conductor carrying the high voltage. The air around the conductor undergoes an electrical breakdown to become conductive (e.g., temporarily) so that charge can leak off of the conductor and into the air. A corona occurs at locations where the strength of the electric field (potential gradient) around a conductor exceeds the dielectric strength of the air.

The ozone generator 101 may use an air source, which may include only ambient air. The air may be provided under stored pressure or a differential pressure in the ozone generator 101. Ambient air may correspond to ozone production in a predetermined concentration range (e.g., 3-6%). Alternatively, an oxygen concentrator may be used to increase the concentration of oxygen in the air source. Pure oxygen may be used. The water vapor content of the air source may be regulated by an air dryer.

The corona discharge may occur in a sealed container of the ozone generator 101. The sealed container may include one or more valves to allow air into the cavity of the sealed container and ozone out of the sealed container.

In another technique, ozone may be produced by ultraviolet light. Such an ozone generator 101 includes a light source that generates a narrow-band ultraviolet light. The narrow-band ultraviolet light may be less than the spectrum of light produced by the sun. Ultraviolet light may produce ozone at a lower concentration (e.g., 1%) than corona techniques. Ultraviolet light ozone generates may exclude both air dryers and oxygen concentrators.

In another technique, ozone may be produced by cold plasma. Such an ozone generator 101 includes a dielectric barrier discharge configured to generate plasma. Pure oxygen gas is supplied to the plasma and the oxygen molecules are split into single atoms, which recombine into groups of three, forming ozone, or O₃. Cold plasma techniques may produce high concentrations of ozone (e.g., 5% or greater) using a small amount of space.

In another technique, an electrolytic ozone generator produces ozone by splitting water molecules. Such an ozone generator 101 includes a water electrolysis device that splits water molecules into H₂, O₂, and O₃. The hydrogen gas, H₂, may be removed to leave oxygen and ozone as the only products of the reaction. Electrolytic ozone generation may produce at higher concentrations (20-30%) than the corona discharge technique. The electrolytic techniques may also avoid nitrogen gases.

The rod 115 extends into the bubble chamber 110. The rod 115 may connect the bubble chamber 110 to the ozone generator 101 and/or the air pump 105. The rod 115 generates or otherwise emits the microbubbles in response to the output of the ozone generator 101 and/or the air pump 105. The rod 115 may include pores having a diameter in the range of 1 nanometer to 20 microns. In one example, the median pore diameter is 2 microns. With this diameter, a relatively low pressure from the air pump 105 is required to push the gases through the pores of the rod 115. In some examples, the rod 115 may be rotating, which is described in more detail below.

After the microbubbles are formed at the surface of the rod 115, the microbubbles detach before attaining a predetermined size (e.g., relatively small size). In the water of the chamber, the microbubbles are attracted to each other due to electrostatic charges. As bubbles collide, the bubbles may combine. Larger bubbles become more buoyant and more likely to rise out of the solution with the water. The chamber may include one or more disruptions to the water to prevent the bubbles from reaching a larger size. In this way, the microbubbles are less likely to coalesce and more likely to remain suspended.

With the microbubbles remaining suspended in the water of the bubble chamber 110, the microbubbles implode. The resulting hydroxyls increase the disinfectant property of the water. The bubble chamber 110 provides the resultant disinfecting water to an output device 120. Examples of the output device 120 are described below.

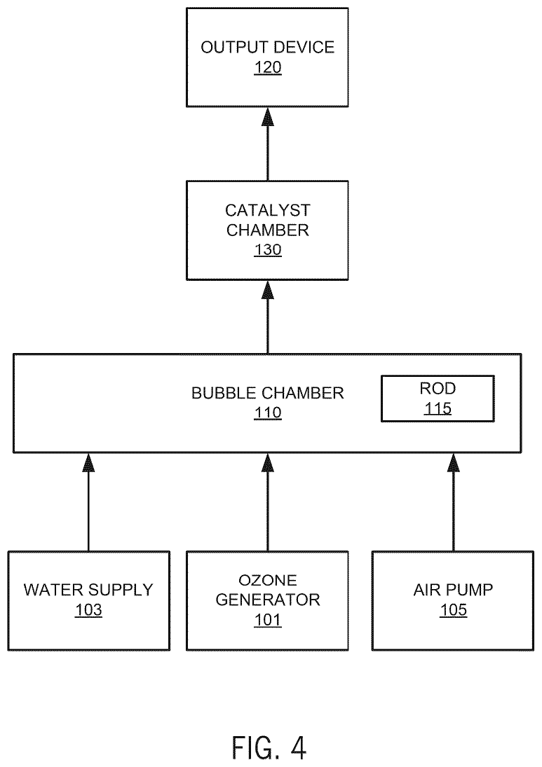

FIG. 4 illustrates an example second embodiment for the disinfection system including a bubble chamber 110 connected to an ozone generator 101, a water supply 103, and an air pump 105. A catalyst chamber 130 connects the bubble chamber 110 to an output device. Additional, different, or fewer components may be included.

The catalyst chamber 130 includes at least one catalyst configured to affect an additional reaction with the microbubbles. The catalyst may include an ultraviolet photo catalyst. Example ultraviolet photo catalysts included titanium dioxide (TiO₂), zinc oxide (ZnO), tin oxide (SnO₂), tungsten oxide (WO₃), cadmium sulfide (CdS), ZnS, CdSe, WS₂, MoS₂, and others.

The catalyst in the catalyst chamber 130 causes one or more reaction with the ozone, oxygen, water, and/or hydroxyl groups as described herein. Products of the reactions included peroxide. The peroxide may be provided to the output device 120.

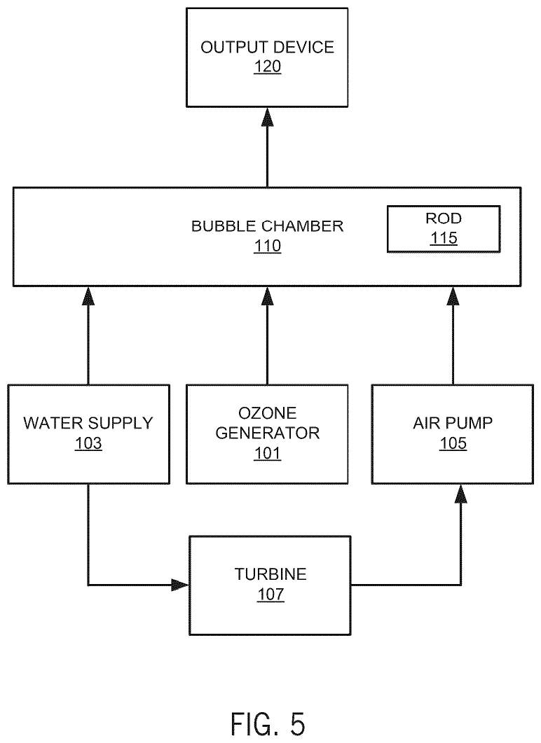

FIG. 5 illustrates an example third embodiment for the disinfection system including a bubble chamber 110 connected to an ozone generator 101, a water supply 103, and an air pump 105. A turbine 107 is connected to the water supply 103 and rotates under a force from the flow of water from the water supply 103. The turbine may rotate an air pump 105. Additional, different, or fewer components may be included.

The turbine 107 is connected to the water supply and configured to translate energy from the water supply to rotate the air pump 105. The air pump 105 is configured to push air into the bubble chamber 110.

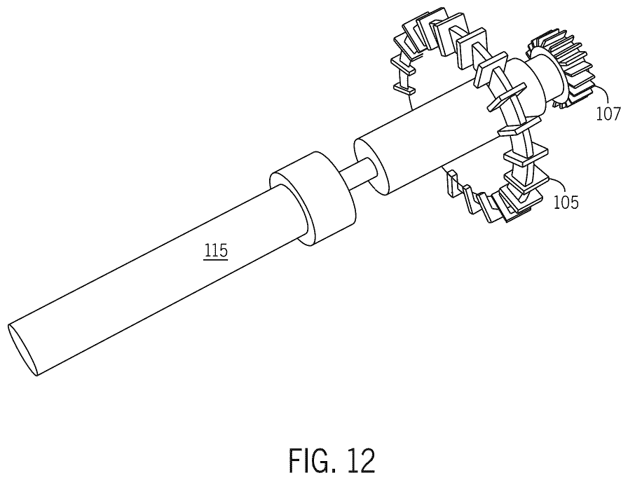

The turbine 107 also rotates the rod 115. The turbine 107 may be integrally formed or otherwise coupled to the rod 115 and/or the air pump 105, as shown in FIG. 12. The flow of water from the water supply 103 to the bubble chamber 110 opposes a rotation of the rod 115.

The rod 115 rotates about an axis. The turbine 107 may rotate about the same axis. The air pump 105 may rotate about the same axis. As described above, the bubble chamber 110 may include one or more disruptions to the water to prevent the bubbles from reaching a larger size. Another example disruption is the angle at which the water path from the water supply 103 meets the rotating rod 115. The water path is directed into the direction of the rotation of the rod 115. A horizontal component of the direction of the water path at the bubble chamber 110 is opposite to a horizontal tangent in a direction of the rotation of the rod 115. In this way, the microbubbles are less likely to coalesce and more likely to remain suspended.

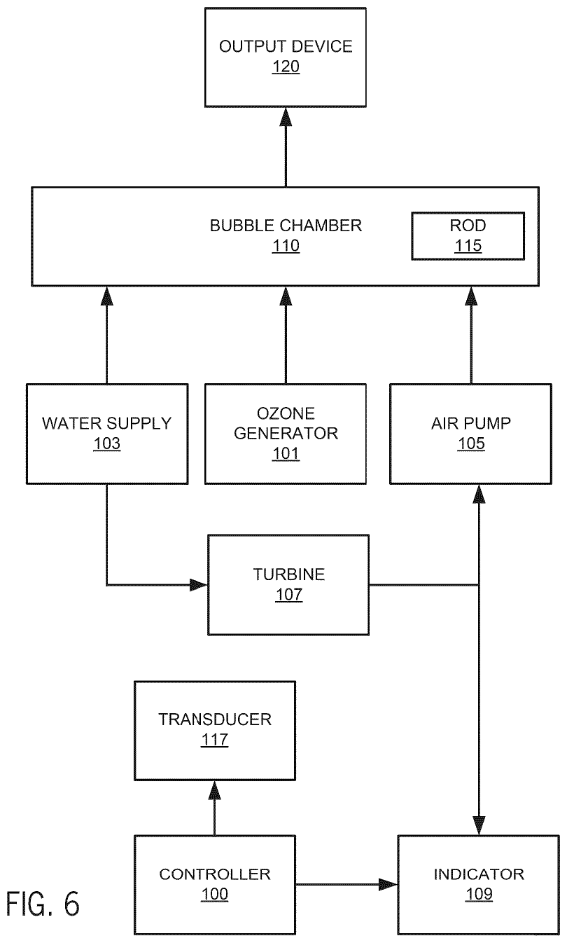

FIG. 6 illustrates an example fourth embodiment for the disinfection system with at least one additional device powered by the turbine 107. The fourth embodiment includes at least a controller 100 and/or a transducer 117. Additional, different, or fewer components may be included.

The transducer 117 is a high frequency generator configured to affect an implosion of the bubbles. The transducer 117 may be a piezoelectric transducer. The transducer 117 may be another disruption to the water to prevent the bubbles from reaching a larger size. In this way, the microbubbles are less likely to coalesce and more likely to remain suspended. In addition, the transducer 117 may facilitate the breakdown of the ozone hydroxyls and/or water at the boundary of the microbubble. In some examples, the ozone is converted to oxygen and oxygen radicals.

The controller 100 is configured to generate a command for the disinfection apparatus. The controller 100 may turn the transducer 117 on and off or set a power level for the transducer 117. The controller 100 may turn the ozone generator 101 on and off or set an output level for the ozone generator 101. The controller 100 may operate the transducer 117 or the ozone generator 101 according to feedback sent from the water consuming device (e.g., user input, user detection). The user may directly provide an input to the disinfection apparatus to operate the transducer 117 or the ozone generator 101. Other inputs such as a mobile device may be used.

The turbine 107 may be omitted. In these examples, another power source is provided to the air pump 105 and/or the rod 115. The controller 100 may turn a motor for rotating the rod 115 on and off or set a speed for the motor of the rod 115. The controller 100 may turn the air pump 105 on and off or set a speed for the air pump 105.

In examples with the turbine 107, the turbine 107 may drive a generator or alternator (e.g., a rotor that rotates within a stator at an air gap). The alternator may include permanent magnets for the exciter of the generator or the main generator or the exciter. The generator may include a wound rotor generator with a permanent magnet exciter, the electrical machine may also act as a motor as well. Other embodiments of electrical machines include permanent magnet brush-type direct current (DC) machines, permanent magnet brushless DC machines, series-wound or universal machines, induction alternating current (AC) machines, synchronous AC machines, synchronous reluctance machines, switched reluctance machines, among others. Any machine may be used as a motor, selectively between a motor and a generator, or entirely as a generator.

A battery may be configured to store energy generated from the turbine 107. The controller 100 may be operable to selectively control charging of the battery from the turbine 107. Energy stored into battery may be provided to one or more devices in the disinfection system. The battery may power a motor for the air pump 104. The battery may power the ozone generator 101. The battery may power a motor for rotating the rod 115.

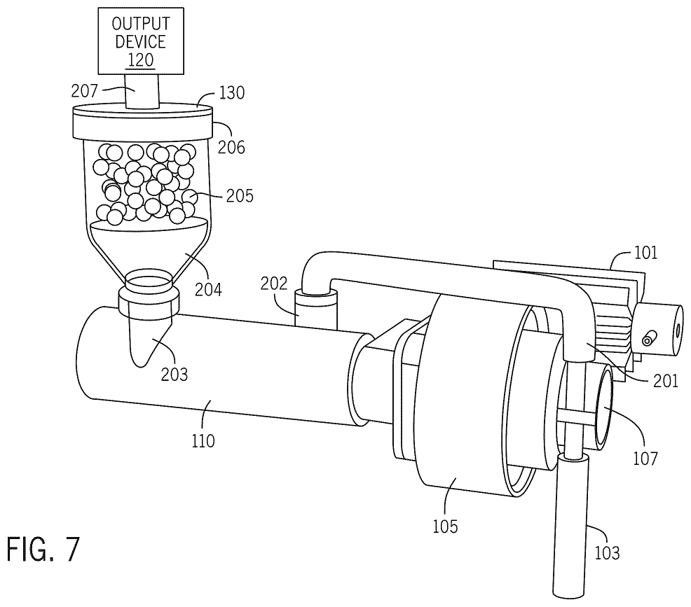

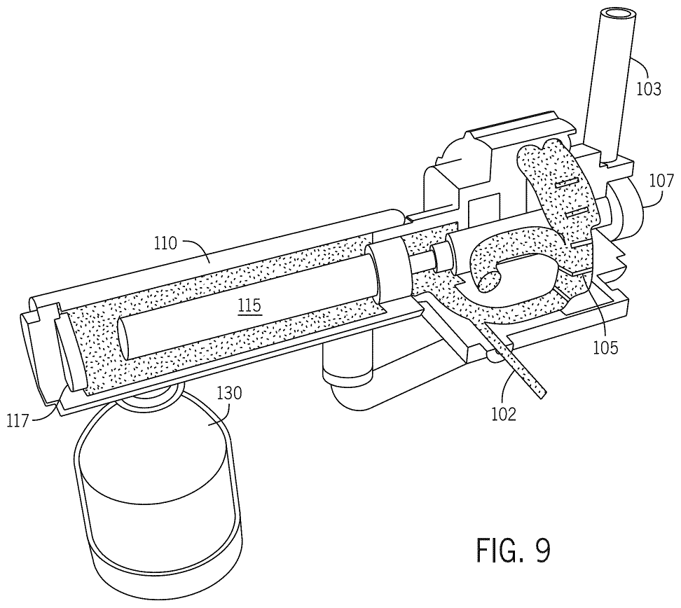

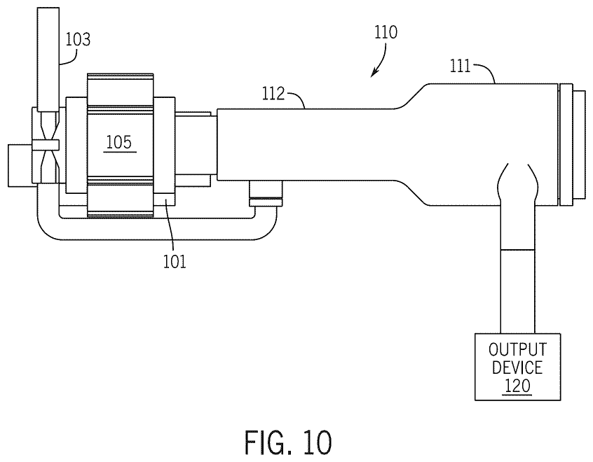

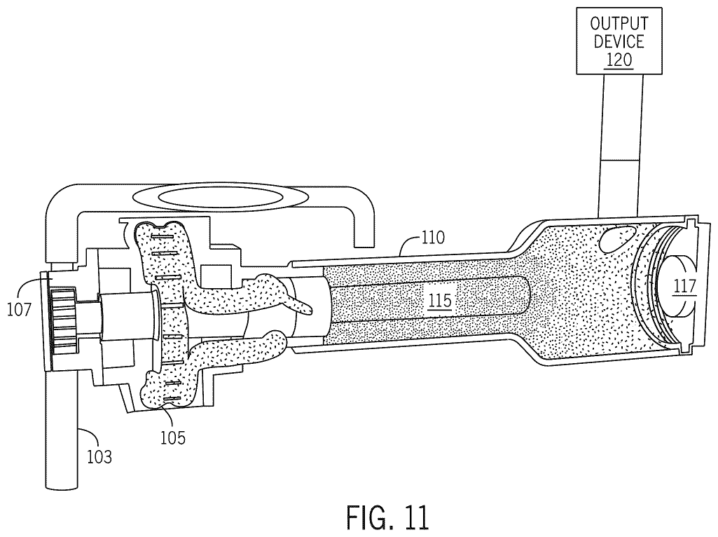

FIGS. 7-9 illustrate example structural components for the disinfection device including the catalyst chamber 130. FIGS. 10 and 11 illustrate example structural components for the disinfection device without the catalyst chamber 130.

The water supply 103 may include a tube that is coupled to the turbine 107. The tube may be connected to the bottom of the turbine and a water output 201 is at the top of the turbine. Another hose or connection tube extends from the water output 201 to the inlet 202 of the bubble chamber 110. The bubble chamber 110 is generally cylindrical, having a circular cross section perpendicular to the flow of water.

An outlet 203 of the bubble chamber 110 is connected to the catalyst chamber 130. The catalyst chamber 130 may include a tapered neck terminating in the downstream direction at a screen or filter 204. The filter 204 prevents the beads 205 from falling into the neck or the bubble chamber 110. A removable lid 206 of the catalyst chamber 230 encloses the beads 205. An output tube 207 connects the catalyst chamber 230 to the output device 120.

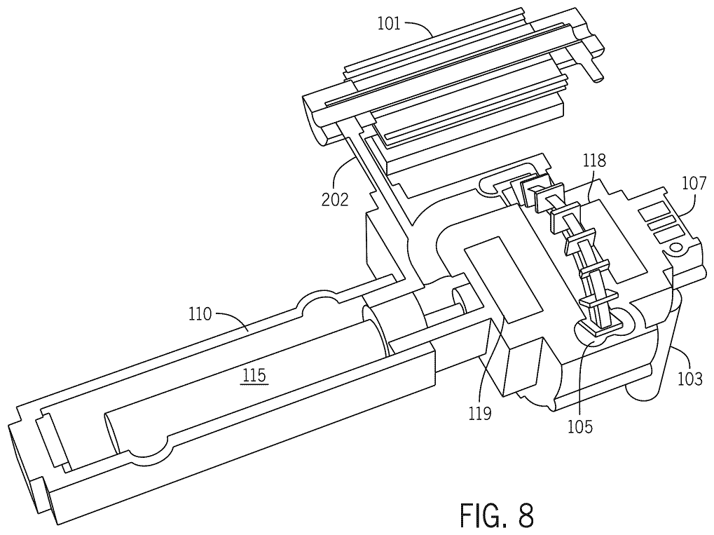

Shown in FIG. 8, a passage from the ozone generator 101 includes a narrow venturi opening 102. The venturi opening 102 creates a differential pressure to draw ozone through the ozone inlet channel. The venturi opening 102 includes a narrowed section of passage having a smaller cross section than a preceding portion of the passage. The narrowed section causes an increase in the velocity of the flow of air and/or water (e.g., ozone). Through the conservation of energy, the increase in the velocity of the flow of air causes a decrease in pressure near the venturi opening 102. The decrease in pressure or differential pressure creates a force that pulls the ozone from the ozone inlet channel.

In any of the following applications for the water disinfection system, a valve may be located between the disinfection system and the output device 120. Alternatively, the valve may be internal to the water consuming appliance. The valve regulates the flow of water from the water disinfection system.

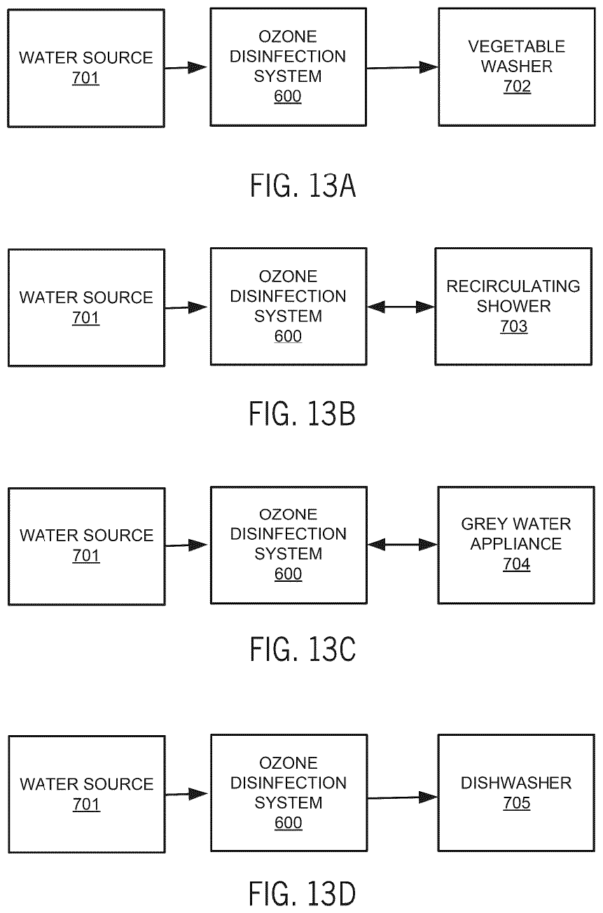

FIGS. 13A-D illustrate example applications for the disinfection system. FIG. 13A includes a water source 701, the ozone disinfection system 600, and a vegetable washer 702. In one embodiment, the vegetable washer 702 may include a tray for lowering vegetables, fruit, or other food items into a cavity connected to the ozone disinfection system 600. The ozone in the cavity washes contaminants from the food items. The ozonated water disinfects the food items.

Alternatively, the vegetable washer 702 includes a sprayer as the output device 120. The sprayer receives line pressure output from the ozone disinfection system and the water is sprayed on fruits and vegetables while the reaction in the water is still active.

FIG. 13B includes a water source 701, the ozone disinfection system 600, and a recirculating shower system 703. The recirculating shower system 703 may include at least one shower outlet and a shower panel fluidly coupled to the shower outlet. The shower panel may be configured to house water recirculation equipment within a showering space (e.g., a corner). In various embodiments, the shower panel may facilitate access to shower control components for ease of installation and/or servicing. The shower panel may also be designed to serve as an aesthetic cover to obscure potentially unsightly shower control components. In various embodiments, the shower control components may include, but are not limited to, one or more pumps, motors, filters, etc. In various embodiments, the shower panel may be removably coupled to the region within the shower via one or more hinges or latches.

Water that exits the recirculating shower 703 may be provided to the ozone disinfection system 600 where it is ozonated for the purposes of disinfection. The water, after disinfection, may be returned to the recirculating shower 703.

FIG. 13C includes a water source 701, the ozone disinfection system 600, and a grey water appliance 704. The grey water appliance 704 may be a water consuming appliance that either provides water to a grey water system or is fed water by the grey water system. Water output from the grey water appliance 704 flows into the ozone disinfection system 600. Optionally, a filter may first filter out contaminants from the water.

FIG. 13D includes a water source 701, the ozone disinfection system 600, and a dishwasher 705. In order to clean the dishwasher 705, the disinfected water may be dispended inside the dishwasher 705 while active reactions are still taking place.

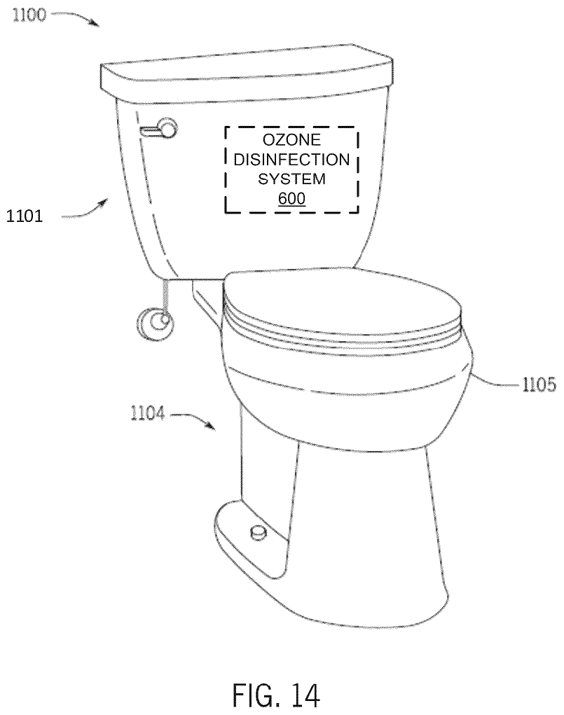

FIG. 14 illustrates a toilet 1100 including the water disinfection system 600. The toilet 1100 may include a tank (e.g., container, reservoir, etc.), shown as a tank 1101, and a pedestal (e.g., base, stand, support, etc.), shown as a pedestal 1104. The tank 1101 may be coupled to, and supported by, the pedestal 1104, which may be positioned on a floor. In some embodiments, the tank 1101 and the pedestal 1104 may be formed together as a single component. In some other examples, the tank 1101 may be mounted nearby on a wall, or within a wall. The tank 1101 is configured to receive water (e.g., via a fill valve of the toilet 1100, etc.) and store the water in between flushes. The pedestal 1104 includes a bowl 1105 and may be configured to receive the water from the tank 1101 to flush contents of bowl into a sewage line. In some embodiments, the pedestal 1104 may be mounted on the wall of a lavatory and the bowl may be configured to receive water from a fluid supply source such as a household water supply.

The bowl 1105 of the pedestal 1104 includes a sump (e.g. a receptacle) and an outlet opening, wherein water and waste is collected in the sump until being removed through the outlet opening, such as when the contents of the bowl are flushed into a sewage line. The toilet 1100 further includes a trapway, and the trapway may be fluidly connected to the bowl via the sump. The trapway fluidly connects the sump to the outlet opening.

The ozone disinfection system 600 may be connected to the fill valve of the toilet. During the flush cycle, or fill cycle, when the fill valve is actuated to fill the tank, water entering the tank first enters the ozone disinfection system 600. In some example, the ozone disinfection system 600 is activated or turned on in response to the fill valve being opened.

In other examples, the controller 100 may trigger the ozone disinfection system 600. The controller 100 may generate commands on a time schedule (e.g., once every predetermined time period or at certain times of day). The controller 100 may send a command to the ozone generator 101 to generate ozone when the tank of the toilet is filled.

The controller 100 may send a command to the ozone generator 101 to generate ozone in response to a user input. For example, a flush cycle may be initiated by the operation of an actuator. The actuator may be a button configured to initiate the flush cycle when depressed (or pulled) a predetermined distance or when touched, a lever configured to activate when rotated a predetermined angular travel, or any suitable device configured to activate based on an input manipulation by a user. In some embodiments, the actuator may be a sensor (e.g., a proximity sensor) and the flush cycle may be automatically initiated (e.g., by a controller) based on sensor data received from the sensor.

The controller 100 may also receive sensor data as feedback for one or more conditions in proximity to the tank 1101 or the toilet. For example, the sensor data may indicate the presence of ozone. The sensor may be an ozone sensor. The ozone sensor may receive air samples and provide one or more tests on the air samples. The ozone sensor may determine a proportion of light that is absorbed by air in the tank 1101 or in the proximity of the toilet 1100. The proportion of light may be indicative of a level of ozone present. The proportion of light may indicate the concentration of ozone. A scrubber within ozone sensor may reset the sensor to test a subsequent air sample.

In the instance of automatic initiation of the flush cycle, the controller 100 may receive sensor data indicative of usage of the toilet. For example, the controller 100 may be in communication with a sensor configured to detect the presence of a user and initiate the flush cycle in response to a user leaving the vicinity of the toilet.

The sensor may include any type of sensor configured to detect certain actions and/or to provide functionality (e.g., dispensing, flushing, etc.). The sensor may include any type of sensor configured to detect certain conditions and/or to provide functionality. Odor sensors, proximity sensors, and motion sensors are non-limiting examples of sensors that may be employed with the systems of this application. Odor sensors, such as volatile organic compound (VOC) sensors, may be employed to detect organic chemicals and compounds, both human made and naturally occurring chemicals/compounds. Proximity sensors may be employed to detect the presence of an object within a zone of detection without physical contact between the object and the sensor. Electric potential sensors, capacitance sensors, projected capacitance sensors, and infrared sensors (e.g., projected infrared sensors, passive infrared sensors) are non-limiting examples of proximity sensors that may be employed with the systems of this application. Motion sensors may be employed to detect motion (e.g., a change in position of an object relative to the object's surroundings). Electric potential sensors, optic sensors, radio-frequency (RF) sensors, sound sensors, magnetic sensors (e.g., magnetometers), vibration sensors, and infrared sensors (e.g., projected infrared sensors, passive infrared sensors) are non-limiting examples of motion sensors that may be employed with the systems of this application.

In another example, the sensor may include a sensor configured to detect a water level. The sensor may include a float sensor, a pressure level sensor, an ultrasonic water level transmitter, a capacitance level sensor (e.g., an RF sensor), and a radar level sensor. Further, an optical sensor may be used to determine a water level.

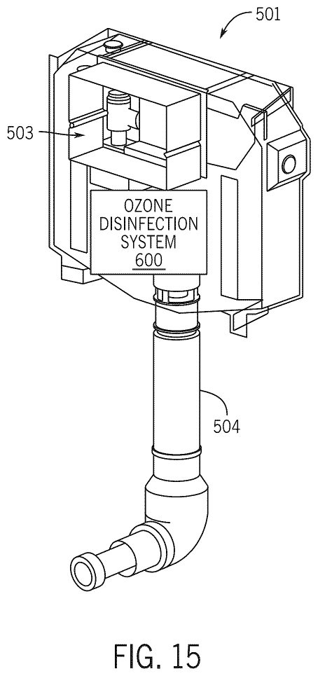

FIG. 15 illustrates an in wall tank 501 including the water disinfection system 600. The in wall tank 501 may include any of the components of the embodiments described herein. The in wall tank 501 is coupled to a toilet (e.g., toilet pedestal) via a water pipe 504. An actuator may be mounted on a wall and coupled to the in wall tank 501 for trigger a flush of the toilet via a user input (e.g., pressure). When the flush is triggered, water is provided from the supply line to the water disinfection system 600 for the water treatment described herein. Water output from the water disinfection system 600 is provided to the toilet via the water pipe 504.

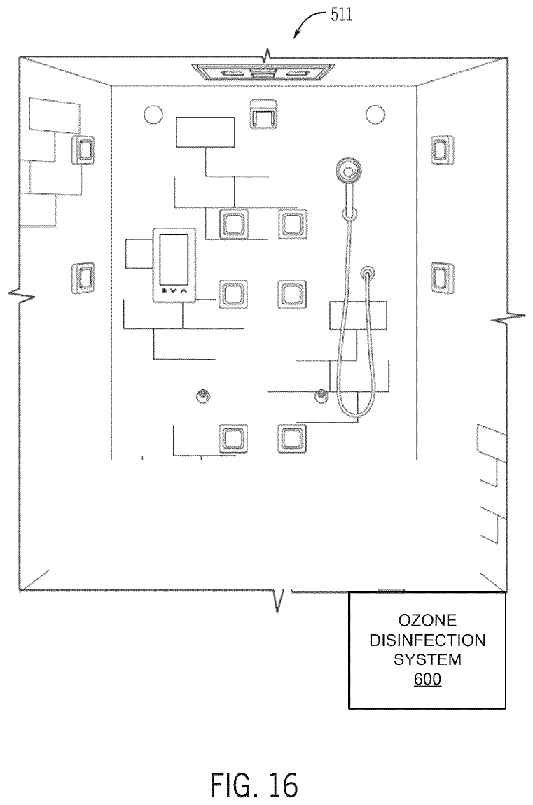

FIG. 16 illustrates a shower 511 including the water disinfection system 600. A valve in the shower 511 opens the supply of water to the water disinfection system 600 for the water treatment described herein. Water output from the water disinfection system 600 is provided to one or more sprayers of the shower 511.

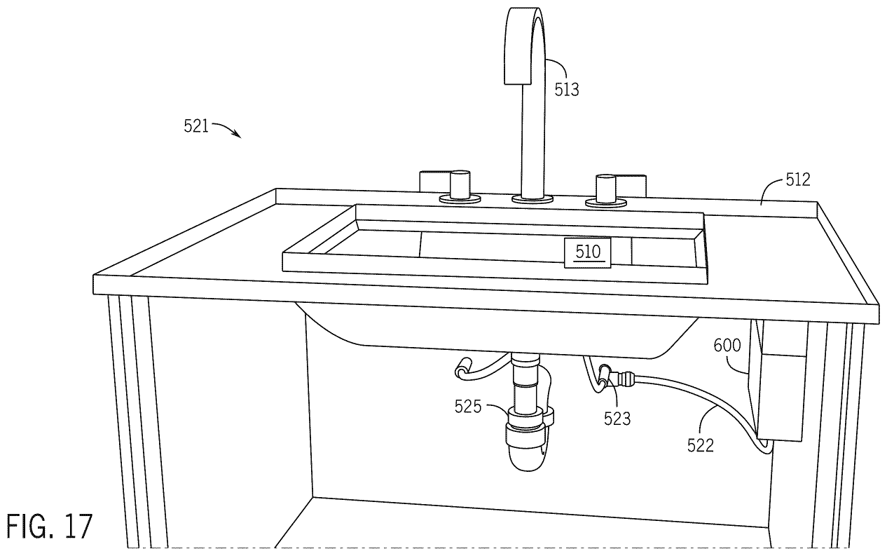

FIG. 17 illustrates a sink 521 including the water disinfection system 600. The sink may include a faucet 513, a basin 510, and a countertop 512. The water disinfection system 600 may be mounted under the countertop 512. A water supply (not shown) enters the water disinfection system 600. An output of the water disinfection system 600 is provided to the faucet 513 via a switch or valve 523. Water leaves the basin 510 via drain 525 and may be recirculated to the water disinfection system 600 in some examples, but may require additional components.

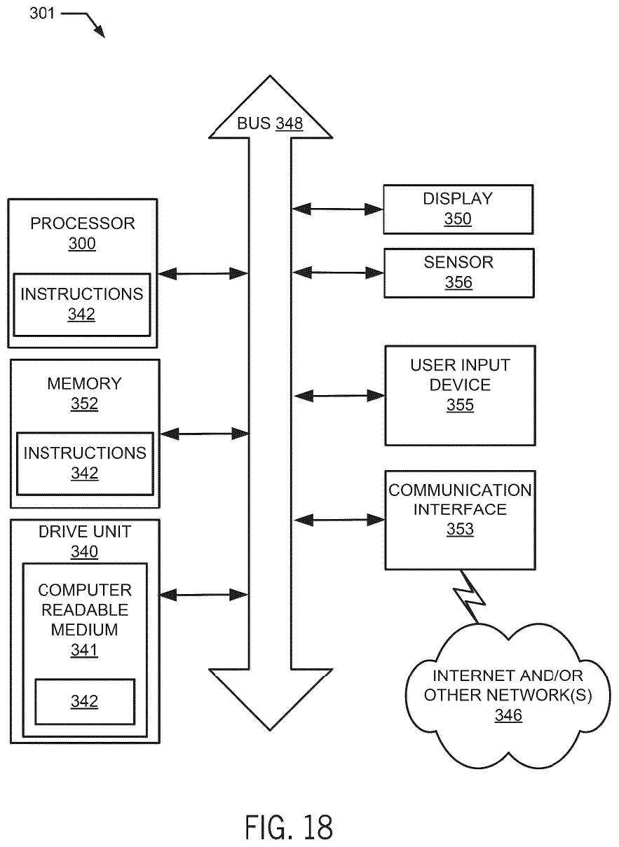

FIG. 18 illustrates an example controller for any of the ozone mitigation systems. The controller 400 may include a processor 300, a memory 352, and a communication interface 353 for interfacing with devices or to the internet and/or other networks 346. In addition to the communication interface 353, a sensor interface may be configured to receive data from the sensors described herein or data from any source for analyzing, ozone, air, or water properties or the operation of the appliances described herein. The components of the control system 400 may communicate using bus 348. The control system 400 may be connected to a workstation or another external device (e.g., control panel) and/or a database for receiving user inputs, system characteristics, and any of the values described herein.

Optionally, the control system 400 may include an input device 355 and/or a sensing circuit in communication with any of the sensors. The sensing circuit receives sensor measurements from as described above. The input device 355 may include a switch (e.g., actuator), a touchscreen coupled to or integrated with, a keyboard, a remote, a microphone for voice inputs, a camera for gesture inputs, and/or another mechanism.

Optionally, the control system 400 may include a drive unit 340 for receiving and reading non-transitory computer media 341 having instructions 342. Additional, different, or fewer components may be included. The processor 300 is configured to perform instructions 342 stored in memory 352 for executing the algorithms described herein. A display 350 may be supported by any of the components described herein. The display 350 may be combined with the user input device 355.

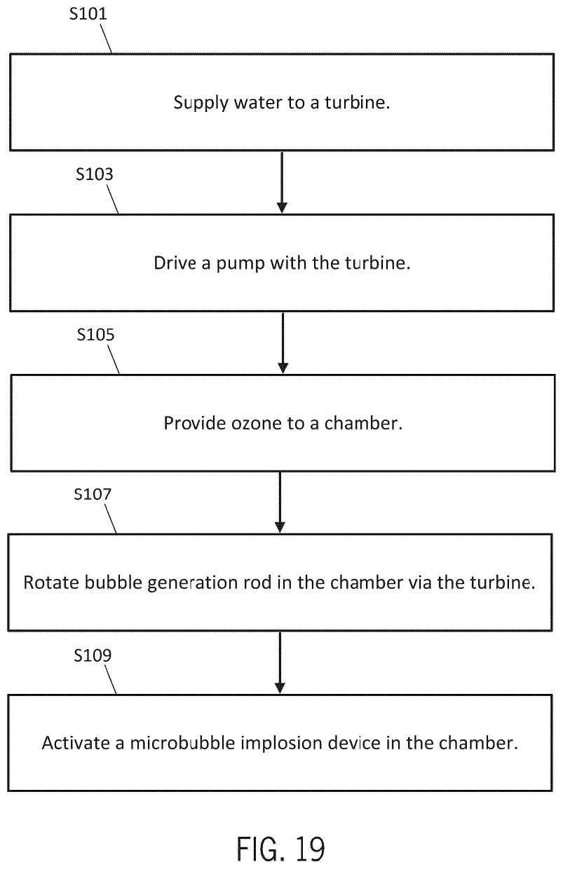

FIG. 19 illustrates a flow chart for the controller of FIG. 18. The acts of the flow chart may be performed by any combination of the control system 400, the network device, or the server. Additional, different or fewer acts may be included.

At act S101, the controller 400 may send a command to actuate a valve to supply water to a turbine. The valve may include an electromagnetic solenoid. The controller 400 may operate an electrical switch to provide power to the electromagnetic solenoid. The controller 400 may send a signal or command to a control circuit for the electromagnetic solenoid include a data for a position of the electromagnetic solenoid.

At act S103, rotation of the turbine may drive a pump. In other words, energy from the movement of the water is transferred from the turbine to a pump. In one example, a shaft extends from the turbine to the pump.

At act S105, act S101, the controller 400 may send a command to activate the ozone generator to provide ozone to a chamber. In some examples, a high voltage source is connected to the ozone generator to operate a corona discharge. The ozone generator may generate microbubbles and/or nanobubbles.

At act S107, the turbine may rotate the bubble generation rod in the chamber with the turbine. The rod may rotate against a flow of water. The rod may emit microbubbles and/or nanobubbles in response to the ozone generator.

At act S109, a microbubble implosion device in the chamber is activated. Example implosion devices included a transducer to facilitate the breakdown of the ozone hydroxyls at the boundary of the microbubbles and/or nanobubbles.

Processor 300 may be a general purpose or specific purpose processor, an application specific integrated circuit (ASIC), one or more programmable logic controllers (PLCs), one or more field programmable gate arrays (FPGAs), a group of processing components, or other suitable processing components. Processor 300 is configured to execute computer code or instructions stored in memory 352 or received from other computer readable media (e.g., embedded flash memory, local hard disk storage, local ROM, network storage, a remote server, etc.). The processor 300 may be a single device or combinations of devices, such as associated with a network, distributed processing, or cloud computing.

Memory 352 may include one or more devices (e.g., memory units, memory devices, storage devices, etc.) for storing data and/or computer code for completing and/or facilitating the various processes described in the present disclosure. Memory 352 may include random access memory (RAM), read-only memory (ROM), hard drive storage, temporary storage, non-volatile memory, flash memory, optical memory, or any other suitable memory for storing software objects and/or computer instructions. Memory 352 may include database components, object code components, script components, or any other type of information structure for supporting the various activities and information structures described in the present disclosure. Memory 352 may be communicably connected to processor 300 via a processing circuit and may include computer code for executing (e.g., by processor 300) one or more processes described herein. For example, memory 298 may include graphics, web pages, HTML files, XML files, script code, shower configuration files, or other resources for use in generating graphical user interfaces for display and/or for use in interpreting user interface inputs to make command, control, or communication decisions.

In addition to ingress ports and egress ports, the communication interface 353 may include any operable connection. An operable connection may be one in which signals, physical communications, and/or logical communications may be sent and/or received. An operable connection may include a physical interface, an electrical interface, and/or a data interface. The communication interface 353 may be connected to a network. The network may include wired networks (e.g., Ethernet), wireless networks, or combinations thereof. The wireless network may be a cellular telephone network, an 802.11, 802.16, 802.20, or WiMax network, a Bluetooth pairing of devices, or a Bluetooth mesh network. Further, the network may be a public network, such as the Internet, a private network, such as an intranet, or combinations thereof, and may utilize a variety of networking protocols now available or later developed including, but not limited to TCP/IP based networking protocols.

While the computer-readable medium (e.g., memory 352) is shown to be a single medium, the term “computer-readable medium” includes a single medium or multiple media, such as a centralized or distributed database, and/or associated caches and servers that store one or more sets of instructions. The term “computer-readable medium” shall also include any medium that is capable of storing, encoding, or carrying a set of instructions for execution by a processor or that cause a computer system to perform any one or more of the methods or operations disclosed herein.

In a particular non-limiting, exemplary embodiment, the computer-readable medium can include a solid-state memory such as a memory card or other package that houses one or more non-volatile read-only memories. Further, the computer-readable medium can be a random access memory or other volatile re-writable memory. Additionally, the computer-readable medium can include a magneto-optical or optical medium, such as a disk or tapes or other storage device to capture carrier wave signals such as a signal communicated over a transmission medium. A digital file attachment to an e-mail or other self-contained information archive or set of archives may be considered a distribution medium that is a tangible storage medium. Accordingly, the disclosure is considered to include any one or more of a computer-readable medium or a distribution medium and other equivalents and successor media, in which data or instructions may be stored. The computer-readable medium may be non-transitory, which includes all tangible computer-readable media.

In an alternative embodiment, dedicated hardware implementations, such as application specific integrated circuits, programmable logic arrays and other hardware devices, can be constructed to implement one or more of the methods described herein. Applications that may include the apparatus and systems of various embodiments can broadly include a variety of electronic and computer systems. One or more embodiments described herein may implement functions using two or more specific interconnected hardware modules or devices with related control and data signals that can be communicated between and through the modules, or as portions of an application-specific integrated circuit. Accordingly, the present system encompasses software, firmware, and hardware implementations.

The light may be a variety of types of light sources such as an ultraviolet (UV) light. The UV light may have a predetermined frequency or wavelength, which may be a range of wavelengths or frequencies for the light emitted from the light. The predetermined frequency or wavelength may be configured to dissipate the ozone in the flow of water. The controller 100 may send commands to the light to start the light or stop the light. The controller 100 may send commands to the light 604 to set the wavelength of the light.

The light may perform both disinfection and ozone dissipation. For example, the light may include ultra-violet germicidal irradiation (UVGI) to kill microbes in the water. The germicidal irradiation may be optimized by a wavelength band of 200 to 280 nanometers (nm). The ozone dissipation may be optimized by a wavelength band of 240 to 315 nm. Thus, the light 604 may be tuned to an overlapping wavelength band of 240 to 280 nm to perform both the germicidal irradiation and the ozone dissipation.

A specific wavelength may be selected based on the structure or gas of an arc lamp or light emitting diode (LED) that is available in the overlapping wavelength band. One example is a low-pressure mercury vapor art lamp at approximately 254 nm.

The illustrations of the embodiments described herein are intended to provide a general understanding of the structure of the various embodiments. The illustrations are not intended to serve as a complete description of all of the elements and features of apparatus and systems that utilize the structures or methods described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

While this specification contains many specifics, these should not be construed as limitations on the scope of the invention or of what may be claimed, but rather as descriptions of features specific to particular embodiments of the invention. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

It is intended that the foregoing detailed description be regarded as illustrative rather than limiting and that it is understood that the following claims including all equivalents are intended to define the scope of the invention. The claims should not be read as limited to the described order or elements unless stated to that effect. Therefore, all embodiments that come within the scope and spirit of the following claims and equivalents thereto are claimed as the invention.

What is claimed is:

1. A disinfection apparatus comprising:
a chamber including a rod;
a water supply configured to supply a flow of water to the chamber;
an air pump configured to supply air to the chamber;
an ozone generator configured to supply ozone to the chamber; and
an output to a plumbing fixture.

2. The disinfection apparatus of claim 1, wherein the plumbing fixture is integrated with or coupled to a toilet, a sink, a lavatory, a shower, or a faucet.

3. The disinfection apparatus of claim 1, wherein the plumbing fixture is integrated with or coupled to a water sprayer.

4. The disinfection apparatus of claim 1, wherein bubbles are formed at the rod in the chamber.

5. The disinfection apparatus of claim 4, wherein the bubbles implode in the chamber.

6. The disinfection apparatus of claim 5, further comprising:
a high frequency generator configured to affect an implosion of the bubbles.

7. The disinfection apparatus of claim 4, further comprising:
a catalyst chamber including at least one catalyst configured to affect an additional reaction with the bubbles.

8. The disinfection apparatus of claim 7, wherein the catalyst chamber includes at least one bead.

9. The disinfection apparatus of claim 1, further comprising:
a turbine connected to the water supply and configured to translate energy from the water supply to rotate the air pump.

10. The disinfection apparatus of claim 9, further comprising:
a controller configured to generate a command for the disinfection apparatus.

11. The disinfection apparatus of claim 10, further comprising:
a battery configured to store energy generated from the turbine, wherein the controller is electrically coupled to the turbine.

12. The disinfection apparatus of claim 5, wherein the plumbing fixture includes a valve and actuation of the valve initiates the flow of water from the water supply.

13. The disinfection apparatus of claim 1, wherein the flow of water to the chamber opposes a rotation of the rod.

14. A disinfection water apparatus comprising:
an air pump;
a chamber including a rod;
a turbine connected to a water supply and configured to translate energy from the water supply to rotate the air pump and the rod;
an ozone supply downstream of the air pump and connected to the chamber; and
a water path from the turbine to the chamber.

15. The disinfection apparatus of claim 14, wherein the rod rotates about an axis.

16. The disinfection apparatus of claim 15, wherein the turbine rotates about the axis.

17. The disinfection apparatus of claim 14, wherein a horizontal component of a direction of the water path at the chamber is opposite to a horizontal tangent in a direction of the rotation of the rod.

18. The disinfection apparatus of claim 14, wherein the turbine turns a generator to generate electricity of an indicator.

19. The disinfection apparatus of claim 14, wherein the turbine turns a generator to generate electricity to charge a battery.

20. A method of supply disinfectant to a plumbing fixture, the method comprising:
supplying water to a turbine;
driving a pump with the turbine;
providing ozone to a chamber;
rotating bubble generation rod in the chamber with the turbine; and
activating a microbubble implosion device in the chamber.
